SEQUENCE LISTING (2 sequences)

SEQ ID NO: 1 - DNA, 1941 bp, African swine fever virus
atggcatcaggaggagctttttgtcttattgctaacgatgggaaggccgacaagattatattggcccaagacttgcttaatagcaggatttctaacattaaaaatgtgaacaaaagttatgggaaacccgaccccgaacccactttgagtcaaatcgaagaaacacatttggttcattttaatgcgcattttaagccttatgttccagtagggtttgaatacaataaagtacgcccgcatacgggtacccccaccttgggaaacaagcttacctttggtattccccagtacggagactttttccatgatatggtgggccaccatatattgggtgcatgtcattcgtcctggcaggatgctccgattcagggcacggcccagatgggggcccatggtcagcttcaaacgtttcctcgcaacggatatgactgggacaaccaaacacctttagagggcgccgtttacacgcttgtagatccctttggaagacctattgtacccggcacaaagaatgcgtaccgaaacttggtttactactgcgaataccccggagaacgactttatgaaaacgtaagattcgatgtaaatggaaattccctggacgaatatagttcggatgtcacaacgcttgtgcgcaaattttgcatcccaggggataaaatgactggatataagcacttggtcggccaggaggtatcggtggagggaactagtggccctctcctatgcaacattcatgatttgcacaagccgcaccaaagcaaacctattcttaccgatgaaaatgatacgcagcgaacgtgcagccataccaacccgaaattcctttcacaacattttcccgagaactctcacaatatccaaacagcaggtaaacaagatattactcctattacggacgcaacgtatctggacataagacgtaatgttcattacagctgtaatggacctcaaacccctaaatactatcagccccctcttgcgctctggattaagctgcgcttttggtttaacgagaacgtgaaccttgctattccctcggtatccattcccttcggcgagcgctttatcaccataaagcttgcatcgcaaaaggatttggtgaatgaatttcctggactttttatacgccagtcgcgttttatacctggacgccccagtagacgcaatatacgctttaaaccatggtttatcccaggagtcattaatgaaatctcgctcacgaataatgaactttacatcaataacctgtttgtaacccctgaaatacacaacctttttgtaaaacgcgttcgattttccctgatacgtgtccataaaacgcaggtgacccacaccaacaataaccaccacgatgaaaaactaatgtctgctcttaaatggcccattgaatatatgtttataggattaaaacctacctggaacatctccgatcaaaatcctcatcaacaccgagattggcacaagttcggacatgttgttaacgccattatgcagcctactcaccacgcagagataagctttcaggatagagatacagctcttccagacgcatgttcatctatatcggatattagccccgttacgtatccgatcacattacctattattaaaaacatttccgtaactgctcatggtatcaatcttatcgataagtttccatcaaagttctgcagctcttacatacccttccactacggaggcaatgcaattaaaacccccgatgatccgggtgcgatgatgattacctttgctttgaagccacgggaggaataccaacccagtggtcatattaacgtatccagagcaagagaattttatattagttgggacacggattacgtggggtctatcactacggctgatcttgtggtatcggcatctgctattaactttcttcttcttcagaacggttcagctgtgctgcgttacagtacctaa

SEQ ID NO: 2 - protein, 646 aa, African swine fever virus
MASGGAFCLIANDGKADKIILAQDLLNSRISNIKNVNKSYGKPDPEPTLSQIEETHLVHFNAHFKPYVPVGFEYNKVRPHTGTPTLGNKLTFGIPQYGDFFHDMVGHHILGACHSSWQDAPIQGTAQMGAHGQLQTFPRNGYDWDNQTPLEGAVYTLVDPFGRPIVPGTKNAYRNLVYYCEYPGERLYENVRFDVNGNSLDEYSSDVTTLVRKFCIPGDKMTGYKHLVGQEVSVEGTSGPLLCNIHDLHKPHQSKPILTDENDTQRTCSHTNPKFLSQHFPENSHNIQTAGKQDITPITDATYLDIRRNVHYSCNGPQTPKYYQPPLALWIKLRFWFNENVNLAIPSVSIPFGERFITIKLASQKDLVNEFPGLFIRQSRFIPGRPSRRNIRFKPWFIPGVINEISLTNNELYINNLFVTPEIHNLFVKRVRFSLIRVHKTQVTHTNNNHHDEKLMSALKWPIEYMFIGLKPTWNISDQNPHQHRDWHKFGHVVNAIMQPTHHAEISFQDRDTALPDACSSISDISPVTYPITLPIIKNISVTAHGINLIDKFPSKFCSSYIPFHYGGNAIKTPDDPGAMMITFALKPREEYQPSGHINVSRAREFYISWDTDYVGSITTADLVVSASAINFLLLQNGSAVLRYST

(19) United States
(12) Patent Application Publication
(10) Pub. No.: US 2024/0181032 A1
(43) Pub. Date: Jun. 6, 2024

(54) VACCINES AND IMMUNOGLOBULINS TARGETING AFRICAN SWINE FEVER VIRUS, METHODS OF PREPARING SAME, AND METHODS OF USING SAME

(71) Applicant: IGY IMMUNE TECHNOLOGIES AND LIFE SCIENCES INC., Thunder Bay, Ontario (CA)

(72) Inventor: Huan Huu Nguyen, Missouri City, TX (US)

(21) Appl. No.: 18/283,163

(22) PCT Filed: Mar. 22, 2022
(86) PCT No.: PCT/IB2022/052621

§ 371 Date: Sep. 20, 2023

Related U.S. Application Data
(60) Provisional application No. 63/164,309, filed on Mar. 22, 2021.

(51) Int. Cl. A61K 39/12; A61K 9/00; A61K 39/00; A61P 31/20; C12N 7/00

(52) U.S. Cl. CPC A61K 39/12; A61K 9/0019; A61P 31/20; C12N 7/00; A61K 2039/505; A61K 2039/5252; A61K 2039/54; A61K 2039/545; A61K 2039/552; C12N 2710/12034; C12N 2710/12051; C12N 2710/12061

(57) ABSTRACT

The present disclosure provides a method of isolating and preparing live African Swine Fever (ASF) viruses (ASFV) and an ASFV vaccine composed of ASF virus particles. ASF viral components, and/or immunosuppressive protein factors. The ASFV vaccine can be used to immunize pigs and wild boars, or can be used to immunize species other than pig or wild boar, such as fowl, bovine, goat, rabbit, donkey or horse, to generate polyclonal immunoglobulins with broad-spectrum specificity to the ASFV. The ASFV-specific immunoglobulins then can be extracted and purified. The ASFV-specific immunoglobulins can provide acute treatment of ASF-infected pigs or wild boars or preventative treatment for pigs or wild boars at risk of ASF, for example that may have been exposed to ASFV or ASFV-infected subjects.

### TECHNICAL FIELD

The present disclosure generally relates to compositions for use in active and/or passive immunization for the treatment and prevention of African Swine Fever (ASF) Virus (ASFV) infection. The present disclosure also relates to methods of isolating and preparing a combination of whole ASF virus particles with ASF individual viral components for use as a vaccine in a swine and/or a non-swine species host for the purpose of generating immunoglobulins specific for ASFV. The immunoglobulins specific for the ASFV that are disclosed herein provide broad-spectrum immunity to pigs and wild boars infected with or susceptible to ASFV infection.

### CROSS REFERENCE TO RELATED APPLICATIONS

This application claims the benefit of U.S. Provisional Application 63/164,309 filed on Mar. 22, 2021, of which is hereby incorporated by reference in its entirety.

### SEQUENCE LISTINGS

The instant application contains Sequence Listings which have been filed electronically in ASCII format and are hereby incorporated by reference in its entirety. Said ASCII copy, created on Mar. 22, 2022, is named Seq_Listings_for_1401870-00015.txt and is 8,548 bytes in size.

### BACKGROUND

ASF is a highly contagious haemorrhagic disease caused by the ASFV. (USDA Surveillance Program, pg 3). ASF affects mammals in the Suidae family, including domestic pigs, feral pigs, and the Eurasian wild boar. (USDA Surveillance Program, pg 3). First identified in East Africa in the early 1900s, the virus spread from indigenous warthogs to domestic pig populations in most sub-Saharan African countries. (Sánchez-Cordón et al., African swine fever: A re-emerging viral disease threatening the global pig industry, 233 Vet. J. 41, 41 (2018)). African warthogs and bush pigs are the natural reservoir hosts for the ASFV, showing few clinical signs and remain persistently infected. (Dixon et al., African swine fever virus evasion of host defences, 266 Virus Res. 25, 25 (2019)). In contrast, infection of domestic pigs, feral pigs, or wild boar results in an acute hemorrhagic fever with high mortality. (Dixon et al., at 25).

The ASFV spread to Europe in the late 1950s and later to South America and the Caribbean. (Sánchez-Cordón et al., at 41). With no effective vaccine, the methods used to control the spread of the virus are limited to quarantine and slaughter of infected and exposed pigs. (Netherton et al., Identification and Immunogenicity of African Swine Fever Virus Antigens, 10 Front. Immun. 1, 1 (2019)). ASF was successfully eradicated from outside Africa in the mid-1990s, but by 2007 the virus had again experienced a second transcontinental spread to Georgia and Eastern Europe. (Sánchez-Cordón et al., at 41). Recently, ASF outbreaks have been reported in China, Vietnam, Mongolia, Cambodia, and Korea (FAO website; ASF situation update). The spread of ASF to China is of particular concern as China is the largest pig producing country in the world. (Netherton et al., at 1).

The ASFV itself is a large, complex double-stranded DNA virus that replicates in the cytoplasm of macrophages, monocytes, and dendritic cells. (Dixon et al., at 25). More than twenty genotypes have been documented and at least eight serotypes have been identified by research groups. (Kolbasov et al., Comparative Analysis of African Swine Fever Virus Genotypes and Serogroups, 21 Emerg. Infect. Dis. 312, 312 (2015)). Traditional inactivated vaccines have been unsuccessful and live-attenuated vaccines have failed to generate the efficacy required. (Sánchez-Cordón et al., at 44). The challenges associated with development of a successful ASF vaccine are thought to be due to a lack of understanding of how the virus modulates the host's response to infection and unidentified protective antigens. (Sánchez-Cordón et al., at 44).

### SUMMARY

The present inventors have developed a method of isolating live ASFV and ASF viral components to make ASFV vaccines comprising comprehensive ASF virus particles, individual ASF viral structural proteins, and ASF viral components involved in exacerbating the infection that include but are not limited to immunosuppressive factors and/or host immune factors, generally derived from ASFV-infected spleen and/or ASFV-infected peripheral blood. Such ASFV vaccine upon gamma irradiation can be used to actively immunize or vaccinate a pig, wild boar or other species susceptible to ASF infection. Additionally or alternatively, live or gamma-irradiated ASFV vaccine can be used to actively immunize or vaccinate a species other than a pig or wild boar, such as a fowl, a bovine, a rabbit, a goat, a donkey, or a horse, to generate polyclonal immunoglobulins with broad-spectrum specificity to the ASFV. In a preferred embodiment, an egg-laying fowl such as a chicken is vaccinated using the ASFV vaccine and the antibodies or antibody fraction then can be extracted and purified from the egg yolk. The egg-laying fowl antibodies produced may be used for the prevention of viral adhesion, viral spread, the treatment of ASF, the prevention of ASF. Antibodies of the IgY isotype from fowl or birds are particularly useful in these applications.

The ASFV-specific immunoglobulins can be administered for acute treatment of an ASFV-infected pig or wild boar. The acute treatment can comprise parenterally and/or orally administering the immunoglobulins, for example by intraperitoneal or intramuscular injection and/or in a food composition. Additionally or alternatively, the immunoglobulins can be administered as a preventative treatment by the same routes of administration. In an embodiment, the ASFV-specific immunoglobulins can be in the form of liquid or a lyophilized powder, reconstituted and then can be intraperitoneally or intramuscularly injected, preferably at an injection dose of about 0.5 to about 1.0 mg per kg body weight twice a week for one or more weeks, for example administered to one or more ASFV-infected or exposed pigs or wild boars. Alternatively, ASFV-specific immunoglobulins can be administered orally, at an oral dose of about 1.0 mg per kg body weight, such as added to the feed once per day for about 5 to about 7 consecutive days, for example administered to one or more ASFV-infected or exposed pigs or wild boars.

In one embodiment disclosed herein is a method of treating ASFV infection in an infected pig or wild boar, the method comprising administering to the infected pig or wild boar an effective amount of a composition comprising immunoglobulins specific against ASF viral components.

Also disclosed herein, the method of treating ASFV infection in an infected pig or wild boar, wherein the composition is administered in an amount that provides a dose of the immunoglobulins specific against ASF viral components that is about 0.5 mg to about 1.0 mg per kg body weight of the infected pig or wild boar.

In another example embodiment, the composition comprising the immunoglobulins specific against ASF viral components is administered for a time period comprising at least once per week or 7 consecutive days.

In one aspect, the composition comprising the immunoglobulins specific against ASF viral components is administered parenterally by intramuscular or intraperitoneal injection.

In another aspect, the composition comprising the immunoglobulins specific against ASF viral components is a food product administered orally.

In another embodiment, is a method of preventing, decreasing incidence of, and/or decreasing severity of ASF viral infection in a pig or wild boar at risk thereof, the method comprising administering to the pig or wild boar an effective amount of a composition comprising immunoglobulins specific against ASF viral components.

In one aspect, the composition is administered in an amount that provides a dose of the immunoglobulins specific against ASF viral components that is about 0.5 to about 1.0 mg per kg of body weight of the pig or wild boar at risk thereof.

In another aspect, the composition comprising the immunoglobulins specific against ASF viral components is administered for a time period comprising at least once per week or 7 consecutive days.

It is also understood that the present disclosure contemplates that the composition comprising the immunoglobulins specific against ASF viral components may be administered parenterally.

In another aspect, the composition comprising the immunoglobulins specific against ASF viral components is a food product administered orally.

Another embodiment disclosed herein is a method of producing ASFV-specific immunoglobulins wherein a ASFV vaccine comprised of whole or fragmented ASF virus particles, ASF viral components, and/or immunosuppressive protein factors, is administered to a non-swine species host for ASFV-specific immunoglobulin production.

In one example embodiment, the host is an egg-laying fowl.

In another example embodiment disclosed herein, is a unit dosage form comprising a therapeutically or prophylactically effective amount of a composition comprising immunoglobulins specific against ASF viral components.

In another embodiment, the composition is a food product formulated for oral administration.

Also disclosed herein is a method of preventing, decreasing incidence of, and/or decreasing severity of ASF viral infection in a pig or wild boar at risk thereof, the method comprising administering to the pig or wild boar an effective amount of an ASFV vaccine composition comprising ASF virus particles, ASF viral components, and/or immunosuppressive protein factors.

In one aspect, the ASF viral components are inactive.

In another aspect, the ASFV vaccine composition is administered parenterally by intramuscular or intraperitoneal injection.

Also disclosed is an example embodiment, wherein the ASFV vaccine composition is administered in an amount that provides a dose of the ASF virus particles, ASF viral components, and/or immunosuppressive protein factors that is about 0.05 mg to about 1.0 mg per pig or wild boar.

In another embodiment, a unit dosage form comprises an effective amount of an ASFV vaccine composition comprising ASF virus particles, ASF viral components, and/or immunosuppressive protein factors.

In one aspect, the ASF viral components are derived from ASF-infected spleen mononuclear cells (SMNCs), ASF-infected peripheral blood and mononuclear cells (PBMCs), and/or ASF-infected primary alveolar macrophages (PAMs).

In another aspect, the ASF virus particles and/or ASF viral components are inactivated.

In another aspect the ASFV vaccine is for use in the treatment and/or prevention of ASF infection in a pig or wild boar at risk thereof.

In one embodiment, immunoglobulins specific against ASF virus particles and ASF viral components for use in the treatment and/or prevention of ASF infection in a pig or wild boar at risk thereof.

It is understood and contemplated herein that the ASFV vaccine may be useful in the preventative treatment of pigs or wild boars against ASF infection. In another embodiment, the ASFV vaccine and the ASFV-specific immunoglobulins may be used in combination and/or administered to a pig or wild boar together in a treatment regimen.

### BRIEF DESCRIPTION OF THE DRAWINGS

FIG. 1 shows example embodiments of a method of making an ASFV vaccine, an embodiment of a method of actively immunizing a pig or wild boar by administering the ASFV vaccine, an embodiment of a method of immunizing or vaccinating a non-swine or non-susceptible species host for producing ASFV-specific immunoglobulins, and an embodiment of a method of passively immunizing a pig or wild boar by administering the ASFV-specific immunoglobulins.

FIGS. 2A-2D show the cytopathic effect of primary alveolar macrophages (PAMs) infected with a live ASFV vaccine composition. FIG. 2A shows a representative microscopy image of a healthy, PAM culture prior to infection with the live ASFV vaccine composition. After infection with the live ASFV vaccine composition, the cytopathic effect on the PAMs in culture can be observed at 3 days after infection (FIG. 2B), 4 days after infection (FIG. 2C), and 7 days after infection (FIG. 2D).

FIGS. 3A and 3B shows example embodiments of active immunization by administering the ASFV vaccine to a pig or wild boar (FIG. 3A) or a non-swine or non-susceptible species host for producing ASFV-specific immunoglobulins (FIG. 3B).

FIG. 4 shows qPCR results for an example embodiment, an absence of ASFV in the blood of hens immunized with live ASFV vaccine.

FIG. 5 shows the effect of exceeding the upper limit of gamma irradiation (i.e., 25 kGy) on ASFV proteins. Gel electrophoresis reveals significantly alter ASFV protein structure (lanes 8-11) following gamma irradiation dose of 25 kGy vs. unirradiated ASFV proteins (lanes 1-6). Molecular ladder (Thang) is shown in lane 7; top molecular marker band is 200 kDa and the lower band is 10 kDa.

FIGS. 6A and 6B show the ASFV p72-specific antibody titers in 3 groups of hens, immunized on day 1, day 14, and day 28 using 2 different ASFV vaccine compositions and saline (no ASFV vaccine) as a control. Eggs laid by immunized hens were collected, immunoglobulins were extracted, and ASFV-specific antibody titers were assessed on day 14 (FIG. 6A) and day 28 (FIG. 6B) using recombinant ASFV major capsid protein p72-coated (ASFV p72; NP_042775.1; SEQ ID NO: 2) enzyme-linked immunosorbent assay (ELISA) plates.

FIG. 7 shows the ASFV-specific antibody titers in 3 groups of hens, immunized on day 1, day 14, and day 28 using 2 different ASFV vaccine compositions and saline (no ASFV vaccine) as a control. Eggs laid by immunized hens were collected starting on day 30, immunoglobulins were extracted, and ASFV-specific antibody titers were assessed using recombinant ASFV major capsid protein p72-coated (SEQ ID NO: 2) ELISA plates.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including,” “containing” and “having” should all be construed to be inclusive, unless such a construction is clearly prohibited from the context. Further in this regard, these terms specify the presence of the stated features but not preclude the presence of additional or further features.

Nevertheless, the compositions and methods disclosed herein may lack any element that is not specifically disclosed herein. Thus, a disclosure of an embodiment using the term “comprising” is (i) a disclosure of embodiments having the identified components or steps and also additional components or steps, (ii) a disclosure of embodiments “consisting essentially of” the identified components or steps, and (iii) a disclosure of embodiments “consisting of” the identified components or steps. Any embodiment disclosed herein can be combined with any other embodiment disclosed herein.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example” and “such as,” particularly when followed by a listing of terms, are merely exemplary and illustrative and should not be deemed to be exclusive or comprehensive.

A “subject” or “individual” is a mammal, preferably a pig or wild boar. As used herein, an “effective amount” is an amount that prevents an infection, treats a disease or medical condition in an individual, or, more generally, reduces symptoms, manages progression of the disease, or attenuates the viral infection for a period of time.

The term “pig” refers to a domestic pig, a wild pig, or a feral pig.

The term “swine” refers to a domestic pig, a wild pig, or a feral pig.

The term “fowl” refers to a wild or domestic egg-laying fowl, such as chicken, duck, swan, goose, turkey, peacock, guinea hen, ostrich, pigeon, quail, pheasant, or dove.

The terms “non-susceptible species” or “non-susceptible host” refer to a species that is not susceptible to ASFV infection or generally, ASF.

The term “immunoglobulin” or “antibody” refers to glycoprotein molecules produced by leukocytes and lymphocytes and are involved in the body's immune system and immune response by specifically recognizing and binding to particular antigens and aiding in their neutralization.

The terms “antigen” or “immunogen” or “hapten” are substances or structures or small molecules that are or are perceived to be foreign to the body and evoke an immune response alone or after forming a complex with a larger molecule. The terms “antigen,” “immunogen,” or “hapten,” are used interchangeably in the present disclosure.

The terms “passive immunity” or “passive immunization” refer to immunity as a result of the introduction of antibodies into the subject from another person, animal, species, or other external source.

The terms “active immunity” or “active immunization” refer to immunity as a result of the natural and/or artificial introduction of antigens into the subject.

The terms “adjuvant” or “immunologic adjuvant” refer to substances that are can added to vaccines to stimulate a subject's immune system's response.

The terms “immunosuppressive protein factors” and/or “host over-reactive immune factors” refer to factors that can include, but are not limited to cytokines (e.g., cytokines of the TNF family), pro-inflammatory cytokines (including, but not limited to TNF-α (e.g., AEP25618), IFN-α (e.g., AFK92985), IL-1β (e.g., NP_001289317), IL-6 (e.g., AFK92986), IL-8 (e.g., NP_999032), IL-12 (e.g., AAA73897 and/or NP_999178), IL-18 (e.g., NP_999162), and RANTES (e.g., NP_001123418)), and/or cytokines involved in the immune response termed the “cytokine storm.” The terms “immunosuppressive protein factors” and/or “host over-reactive immune factors” can be used interchangeably herein, and generally refer to factors that evade the innate and/or adaptive immune responses. “Immunosuppressive protein factors” and/or “host over-reactive immune factors” can be derived from ASFV infected lung tissue, spleen tissue and/or ASFV infected peripheral blood.

The terms “treatment” and “treat” include both prophylactic or preventive treatment (that prevent and/or slow the development of a targeted pathologic condition, infection, disorder, or disease) and curative, therapeutic or disease-modifying treatment, including therapeutic measures that cure, slow down, lessen symptoms of, and/or halt progression of a diagnosed pathologic condition, infection, disorder, or disease; and treatment of subjects at risk of contracting a disease or infection or suspected to have contracted a disease or infection, as well as subjects who are ill or have been diagnosed as suffering from a pathologic condition, infection, disorder, or disease. The terms “treatment” and “treat” do not necessarily imply that a subject is treated until total recovery. The terms “treatment” and “treat” also refer to the maintenance and/or promotion of health in an individual not suffering from a pathologic condition, infection, disorder, or disease but who may be susceptible to the development of a pathologic condition, infection, disorder, or disease. The terms “treatment” and “treat” are also intended to include the potentiation or otherwise enhancement of one or more primary prophylactic or therapeutic measures. As non-limiting examples, a treatment can be performed by a doctor, a healthcare professional, a veterinarian, a veterinarian professional, an animal handler, or another human.

The term “unit dosage form,” as used herein, refers to physically discrete units suitable as unitary dosages for subjects, each unit containing a predetermined quantity of the composition disclosed herein in amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

The term “sterile” is understood to mean free from any bacteria or other living microorganisms.

The term “pharmaceutically acceptable” as used herein refers to substances that do not cause substantial adverse allergic or immunological reactions when administered to a subject.

All percentages expressed herein are by weight of the total weight of the composition unless expressed otherwise. When reference herein is made to the pH, values correspond to pH measured at about 25° C. with standard equipment. “Ambient temperature” or “room temperature” is between about 15° C. and about 25° C., and ambient pressure is about 100 kPa.

The term “mM”, as used herein, refers to a molar concentration unit of an aqueous solution, which is mmol/L. For example, 1.0 mM equals 1.0 mmol/L.

The terms “substantially no,” “essentially free” or “substantially free” as used in reference to a particular component means that any of the component present constitutes no more than about 3.0% by weight, such as no more than about 2.0% by weight, no more than about 1.0% by weight, preferably no more than about 0.5% by weight or, more preferably, no more than about 0.1% by weight.

The terms “food,” “food product” and “food composition” mean a product or composition that is intended for ingestion by an animal, and provides at least one nutrient to the animal. Preferred embodiments of a food product include at least one of a protein, a carbohydrate, a lipid, a vitamin, or a mineral. Food products may include macronutrients and/or micronutrients.

The terms “immunize” or “vaccinate” within this disclosure are used interchangeably.

### Embodiments

### ASFV Vaccine

The present disclosure generally relates to an ASFV vaccine comprising a combination of whole live ASFV particles and naturally expressed ASFV components, as well as immunosuppressive protein factors and/or host over-reactive immune factors, optionally diluted in sterile buffer, for example diluted to about 10% in sterile saline buffer. The ASFV vaccine can be used to actively immunize or vaccinate a non-susceptible species host for the production of ASFV-specific immunoglobulins. A non-susceptible species host can be a non-swine mammal host, for example, a fowl, horse, bovine, donkey, goat, or rabbit.

Another embodiment relates to an ASFV vaccine comprising a combination of whole and/or fragments of ASFV particles and naturally expressed ASFV components, optionally diluted in sterile buffer. The ASFV vaccine can be used to actively immunize or vaccinate a non-susceptible species host for the production of ASFV-specific immunoglobulins.

Another aspect of the present disclosure generally relates to a method of producing the ASFV vaccine. In a preferred embodiment, the ASFV antigens, as well as immunosuppressive protein factors and/or host over-reactive immune factors, are obtained from an ASF-infected pig or wild boar. In one embodiment, blood can be withdrawn from the ASF-infected pig or wild boar and collected into a blood collection tube with anti-coagulant. The blood collection tubes can be centrifuged, for example at about 1,500×g for about 15 minutes at about 4ºC, to obtain buffy coat. Alternatively, the plasma-containing peripheral blood and mononuclear cells (PBMCs) can be separated from the blood by standard gradient centrifugation on Ficoll or other method known to a person of skill in the art. In addition, any red blood cells (RBCs) can be lysed using a solution comprising about 0.83% NH₄Cl or by any other method known to a person of skill in the art.

The collected and/or separated PBMCs can be disrupted and/or lysed by one or more freeze-thaw cycles, for example placed in dry ice ethanol bath (about −72° C.), for a first predetermined time period and then placed at room temperature for a second predetermined time period. This process can be repeated one or more times. The disrupted PBMCs can be centrifuged in a second centrifugation step, for example at about 800×g for about 15 minutes at about 4° C. The supernatant preferably contains whole ASF virus particles, ASF viral components, immunosuppressive protein factors and/or host over-reactive immune factors, and can be collected and diluted one or more times, for example 10 times, with a buffer, such as sterile saline buffer, at a predetermined pH. The resulting ASFV vaccine can be stored at a temperature below room temperature in one or more portions, for example at or below about −20° C. in about 1 ml aliquots. In one example embodiment, the protein content and/or virus titer in the supernatant can be assessed prior to freezing and storing.

In another embodiment, the ASFV vaccine can be obtained from an ASFV-infected lymphoid organ such as a spleen. The spleen can be harvested from an ASFV-infected pig or wild boar and dissected into a plurality of tissue sections. The ASFV-infected spleen tissue not only contains ASF virus particles and/or ASF viral components, but also immunosuppressive protein factors and/or host over-reactive immune factors. Preferably the dissection is immediately after harvesting. The tissue sections can be added to a buffer and minced using metal mesh or homogenized on ice. The homogenized tissue mixture can be centrifuged to generate a single cell suspension, for example centrifuged at about 800×g, at a predetermined time and a predetermined temperature, for example about 15 minutes at about 4° C. The single cell suspension may contain RBCs and spleen mononuclear cells (SMNCs). The RBCs can be lysed using a solution comprising about 0.83% NH₄Cl or by any other method known to a person of skill in the art. SMNCs can be collected and lysed by any method known to a person of skill in the art. Cell debris can be removed by centrifugation and the supernatant can be collected.

The supernatant preferably contains whole ASF virus particles, ASF viral components, immunosuppressive protein factors, and SMNCs can be collected by Ficoll gradient centrifugation. The supernatant and SMNCs can be collected and subjected to one or more freeze-thaw cycles, wherein the mixture can be reduced to a low temperature, for example placed in dry ice ethanol bath (about)−70° ° C., for a first predetermined time period and then placed at room temperature for a second predetermined time period. The mixture of supernatant and disrupted SMNCs can be centrifuged at about 800×g for about 15 minutes at about 4° C. The supernatant can be collected and diluted one or more times, for example 10 times, with a buffer, such as sterile saline buffer, at a predetermined pH. The resulting ASFV vaccine can be stored at a temperature below room temperature in one or more portions, for example at or below −20° ° C., preferably about −70° C., in about 1 ml aliquots. In another example embodiment, the protein content and/or virus titer in the supernatant can be assessed prior to freezing and storing.

In another embodiment, the ASFV vaccine can be obtained from the ASFV vaccine can be obtained from other ASFV-infected tissues or organs such as the lungs. The lungs can be harvested from an ASFV-infected pig or wild boar and dissected into a plurality of tissue sections. The ASFV-infected lung tissue not only contains ASF virus particles and/or ASF viral components, but also immunosuppressive protein factors and/or host over-reactive immune factors. Preferably the dissection is immediately after harvesting. The tissue sections can be added to a buffer and minced using metal mesh or homogenized on ice. The homogenized tissue mixture can be centrifuged to generate a single cell suspension, for example centrifuged at about 800×g, at a predetermined time and a predetermined temperature, for example about 15 minutes at about 4° C. A cell suspension can be prepared and lysed using methods known to a person of ordinary skill in the art to yield a supernatant that preferably contains whole ASF virus particles, ASF viral components, and immunosuppressive protein factors and/or host-over-reactive immune factors.

The immunosuppressive protein factors and/or host-over-reactive factors, such as TNF-α, IFN-α, IL-1β, IL-6, IL-8, IL-12, IL-18, and/or RANTES can vary in quantity depending on disease progression and/or tissue type. The ASFV vaccine composition is standardized using the total amount of total proteins.

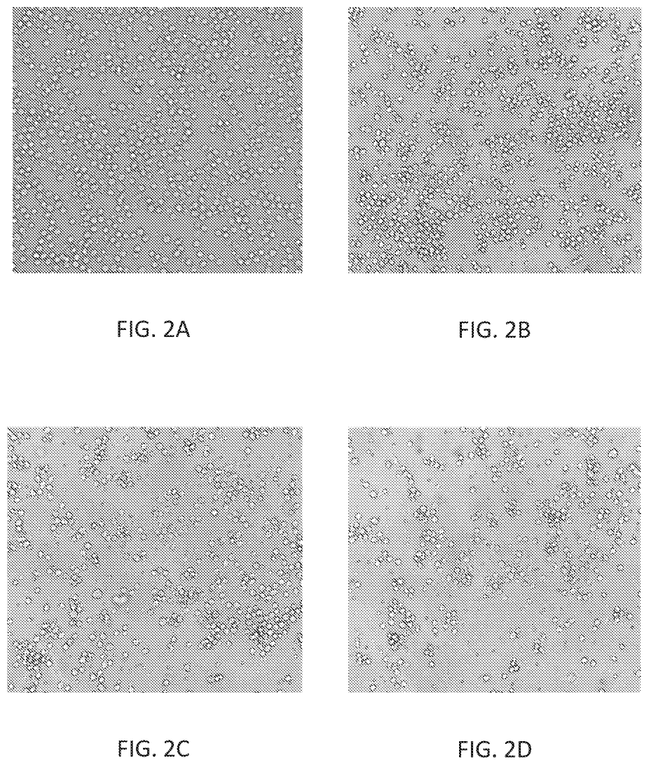

In another embodiment, fresh primary alveolar macrophages (PAMs) were collected from healthy pigs and plated in cell culture flasks for overnight culture with complete medium containing fetal bovine serum (FBS; FIG. 2A). After about 24 hours, live ASFV stock can be added to the culture. The ASF-infected PAMs can be cultured until at least about a 75% cytopathic effect was observed in the culture, for example after about five to about seven days post-ASFV infection (FIGS. 2B, 2C, and 2D). PAMs and the culture supernatant can be harvested, collected and can be subjected to one or more freeze-thaw cycles, wherein the PAM mixture can be reduced to a low temperature, for example placed in dry ice ethanol bath (about −70° C.), for a first predetermined time period and then placed at room temperature for a second predetermined time period. The mixture of supernatant and disrupted PAMs can be centrifuged at about 800×g for about 15 minutes at about 4° C. The supernatant can be collected and diluted one or more times, for example 10 times, with a buffer, such as sterile saline buffer, at a predetermined pH. The resulting ASFV vaccine can be stored at a temperature below room temperature in one or more portions, for example at or below −20° C., preferably about −70° C., in about 1 ml aliquots. In another example embodiment, the protein content and/or virus titer in the supernatant can be assessed prior to freezing and storing.

In a preferred embodiment, the ASFV vaccine composition comprises a protein mixture, ASF virus particles, ASF viral components, and immunosuppressive protein factors/host-over-reactive factors from one or more than one of the following, SMNCs, PBMCs, and/or PAMs.

It is understood and contemplated herein that the ASFV vaccine composition contains a wide range of naturally synthesized, ASFV antigens (i.e., comprehensive ASFV proteins). It is understood that the proteins or antigens that may comprise the ASFV vaccine composition, may include the full, in-tact ASFV proteins and/or may also comprise parts or segments of the disclosed ASFV proteins.

It is also understood that if desired, a particular genotype or serotype of the ASFV can be selected for producing the ASFV vaccine composition, by first testing the infected pig. Additionally or alternatively, the ASFV methods of treatments disclosed herein can provide cross-protection against closely related virus strains, ASFV genotypes, and/or ASFV serotypes.

Also disclosed herein are methods for inactivating the ASFV vaccine composition prior to use. In one example embodiment, the ASFV vaccine composition may be irradiated using gamma irradiator at a dose range of about 2 kGy to about 20 kGy. At a dose of about 15 kGy or about 20 kGy, ASFV DNA is damaged while viral morphology and viral protein integrity are generally preserved.

Additionally or alternatively, a non-irradiated ASFV vaccine can be used to vaccinate or immunize non-swine mammal host, such as a fowl, horse, bovine, donkey, goat, or rabbit, such as for generating ASFV-specific immunoglobulins.

### ASFV-Specific Immunoglobulins

Another aspect of the present disclosure generally relates to the method of immunizing or vaccinating a non-susceptible species host to generate ASFV-specific immunoglobulins. An ASFV vaccine comprising whole ASF virus particles, ASF viral components, immunosuppressive protein factors, and host over-reactive immune factors, for example an aliquot (e.g., about 1 ml) of about 10% ASFV vaccine in sterile saline buffer, can be thawed to a predetermined temperature, vortexed and injected intramuscularly into a non-swine mammal host, such as a fowl, horse, bovine, donkey, goat, or rabbit. Following the initial and optional re-immunizations, a sample of the hosts' venous blood can be collected by various methods known by a person of ordinary skill in the art.

In a preferred embodiment, the anti-ASFV immunoglobulins are IgY antibodies produced by an immunized or vaccinated egg-laying fowl, such as a chicken. An ASFV vaccine comprising whole ASF virus particles, ASF viral components, and immunosuppressive protein factors, for example an aliquot (e.g., about 1 ml) of about 10% ASFV vaccine in sterile saline buffer, can be thawed to room temperature, vortexed and injected intramuscularly into the egg-laying fowl. Preferably, the ASFV vaccine is split into equal fractions (about 100 μg protein content/fraction), with one fraction injected into the left breast of the hen and the second fraction injected into the right breast of the hen, optionally in approximately equal volume amounts such as about 500 ml into the right breast and about 500 ml into the left breast. Additionally or alternatively, the ASFV vaccine can be emulsified with complete Freund's adjuvant (CFA), in about a 1:1 ratio, before injecting the ASFV vaccine into the hen. In another embodiment, subsequent immunizations may include ASFV vaccine compositions comprising about a 1:1 solution of ASFV vaccine and incomplete Freund's adjuvant (IFA).

The hen can be re-immunized following the initial immunization, for example about 7 days following the initial immunization and/or about 14 days following the initial immunization and/or about 28 days following the initial immunization. After initial immunization and any re-immunization (e.g., about twenty-seven days after the initial immunization), eggs laid by the immunized hen can be collected for one or more days for purification of antibodies IgY. Alternatively, the eggs can be continuously collected during the immunization period. The IgY antibodies can be obtained from the collected egg yolks via water-soluble fractions. One or more egg yolks can be pooled and diluted about 10 times with cooled 3 mM HCl to give the suspension a final of about pH of 5 (adjusted with approximately 10% acetic acid). The suspension can be frozen, for example, overnight at about −20° ° C. After thawing to a predetermined temperature, the mixture can be centrifuged at about 13,000×g for about 15 minutes at approximately 4° C. and the supernatant containing the IgY immunoglobulins can be collected. The IgY immunoglobulins can be further purified by various precipitation methods known to a person of ordinary skill in the art, such as using ammonium sulfate or bio-compatible sodium chloride (See Hodek, P. et al., Optimized Protocol of Chicken Antibody (IgY) Purification Providing Electrophoretically Homogenous Preparations, 8 Int. J. Electrochem. Sci. 113, 113-124 (2013)). Alternatively, the IgY immunoglobulins can be obtained from the egg white fraction.

In some embodiments the ASFV-specific immunoglobulin composition comprises the yolk of the egg, or any IgY antibody-containing fraction thereof. The yolk is the preferable portion of the egg, as the yolk typically contains much higher concentrations of IgY than does the white. However, the white may contain concentrations of IgY sufficient for some applications.

In some embodiments of the antibody composition, the IgY is concentrated, isolated, or purified from the constituent of the egg. This can be accomplished by a variety of methods, for example, methods known by a person of ordinary skill in the art. If desired, the titer of IgY antibodies can be determined by immunoassay, for example ELISA.

In some embodiments of the antibody composition, the composition is made by the method comprising obtaining an egg laid by a fowl previously actively vaccinated against ASFV and separating the antibody fraction from a yolk of the egg. The fowl is preferably a domesticated fowl. The domesticated fowl may be chicken, duck, swan, goose, turkey, peacock, guinea hen, ostrich, pigeon, quail, pheasant, dove, or other domesticated fowl. The domesticated fowl is preferably a chicken. The domesticated fowl is more preferably a domesticated chicken raised primarily for egg or meat production.

In some embodiments of the antibody composition, the antibody composition is made by a method comprising actively vaccinating a hen against ASFV, collecting eggs from the hen after an immunization period, and separating the antibody fraction from a yolk of the egg. Optionally, collecting eggs from the hen can occur continuously after the immunization period.

Further methods of producing IgY with a specific target are known to those skilled in the art, although these methods are not known to have been previously successfully used to produce antibodies to ASFV. The antibodies disclosed in this section are suitable for use in any of the methods and compositions described in this disclosure.

It has been discovered that IgY antibodies from fowl eggs are generally cost-effective and a plentiful source of viral adhesion inhibitors (i.e. immunoglobulins). Such antibodies bind to the surface of an antigen-bearing virus (such as ASFV), thus preventing the initial stages of contact between the virus and a potential host cell. Other IgY antibodies bind to internal viral proteins, expressed on the surface of infected cells, further reducing and/or preventing virus spread from infected cells to uninfected cells. As explained elsewhere in this disclosure, preventing the initial stages of adhesion between a virus and a host cell, as well as inhibiting virus spread, has numerous applications, including treatment of viral disease and prevention of viral disease.

In some embodiments of the inhibitor, the inhibitor comprises a constituent of a fowl egg, wherein the fowl egg comprises an adhesion-inhibiting and effective amount of IgY specific for ASFV. Additionally or alternatively, the inhibitor comprises a constituent of a fowl egg, wherein the fowl egg comprises an effective amount of IgY specific for ASFV to inhibit the virus spread. The constituent of the fowl egg may be any constituent described as appropriate antibody compositions in this disclosure.

Methods are provided for preventing viral adhesion to a cell and/or virus spread. The first step in the infection of a cell by a virus is contact and adhesion between virus and cell. Although this step is critical to the establishment of infection, methods of preventing infection at this early stage are few. More typically viral infection is countered using techniques such as active vaccination, which causes the body to produce antibodies that neutralize the virus. If active vaccination is not feasible, most often viral disease is merely treated symptomatically. The methods described here offer an effective means to prevent this early step in the infection process without requiring administration well in advance of the subject's exposure to the pathogen, as is required by active vaccination.

Antibodies can function to prevent adhesion between virus and cell by binding to the virus and interfering with the ability of the virus to bind its target membrane receptor. In addition, antibodies can function to prevent virus spread from infected cells to uninfected cells by binding to viral proteins expressed on surface of infected cells. Avian antibodies (such as IgY) have distinct advantages over mammalian antibodies in this application, particularly when the subject is a mammal. As stated above, the advantages of IgY antibodies include that IgY antibodies as compared to mammalian antibodies are more specific, more stable, and cause fewer unwanted forms of immune response. IgY antibodies can also be easily and cheaply obtained from eggs.

In one embodiment of the method, the method comprises administering to an subject an adhesion-inhibiting effective amount of a viral adhesion inhibitor. The viral adhesion inhibitor can be any embodiment of the ASFV-specific immunoglobulin composition disclosed herein. In some embodiments of the method, the viral adhesion inhibitor comprises a constituent of a fowl egg, the constituent comprising an adhesion-inhibiting effective amount of IgY-specific for ASFV. The constituent may be any constituent disclosed herein as an appropriate antibody composition.

In some embodiments of the method, the ASFV-specific immunoglobulin composition is a pharmaceutical comprising the contents of a fowl egg, the contents of the fowl egg comprising an effective amount of IgY-specific for ASFV. The pharmaceutical may comprise additional components as discussed herein. The pharmaceutical may be administered by any method known in the art or as described herein.

### Methods of Treatment

Yet another aspect of the present disclosure generally relates to a pharmaceutically acceptable compositions of ASFV vaccines and ASFV-specific immunoglobulins that can be administered to ASFV-infected or exposed pigs or wild boars. Additionally or alternatively, the ASFV vaccine may be administered to a non-swine mammal host, as previously described.

In one embodiment, the ASFV vaccine and/or the ASFV-specific immunoglobulins are in the form of compositions, such as but not limited to, pharmaceutical compositions. The compositions disclosed may comprise one or more of such compositions disclosed above, in combination with a pharmaceutically acceptable carrier. Examples of such carriers and methods of formulation may be found in Remington: The Science and Practice of Pharmacy (20th Ed., Lippincott, Williams & Wilkins, Daniel Limmer, editor). To form a pharmaceutically acceptable composition suitable for administration, such ASFV-specific immunoglobulins compositions will contain a therapeutically effective amount of an antibody. The therapeutically effective amount of the antibody may be an adhesion inhibiting effective amount and/or an amount effective to generate passive immunity in the subject (i.e., pig or wild boar). Additionally or alternatively, to form a pharmaceutically acceptable composition suitable for administration, such ASFV vaccine compositions will contain a therapeutically effective amount of an ASFV antigen (e.g., ASFV virus particles and/or ASF viral components). The therapeutically effective amount of the irradiated ASFV antigens may be an amount effective to generate protective immunity in the subject (i.e., pig or wild boar).

The pharmaceutical compositions of the disclosure may be used in the treatment and prevention methods of the present disclosure. Such compositions are administered to a pig or wild boar in amounts sufficient to deliver a therapeutically effective amount of the ASFV-specific immunoglobulins or ASFV vaccine so as to be effective in the treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. Exemplary routes of administration include, but are not limited to, intraperitoneal, intramuscular, subcutaneous, intravenous, topical, epicutaneous, oral, intraosseous, intranasal. Oral administration of the ASFV-specific immunoglobulins may be achieved by adding to the subject's feed (solid or liquid).

The compositions of the present disclosure may be administered only one time to the subject or more than one time to the subject. Furthermore, when the compositions are administered to the subject more than once, a variety of regimens may be used, such as, but not limited to, one per day, once per week, once per month or once per year. The compositions may also be administered to the subject more than one time per day. The therapeutically effective amounts and appropriate dosing regimens of the ASFV-specific immunoglobulin composition and/or the ASFV vaccine composition may be identified by routine testing in order to obtain optimal activity, while minimizing any potential side effects. The ASFV-specific immunoglobulin composition and the ASFV vaccine composition may be administered individually, to separate subjects. Additionally or alternatively, the ASFV-specific immunoglobulin composition and the ASFV vaccine composition may be co-administered in various treatment regimens to an individual subject in need thereof. In addition, co-administration or sequential administration of other agents may be desirable.

The compositions of the present disclosure may be administered systemically, such as by intraperitoneal, intravenous, or intramuscular administration.

The compositions of the present disclosure may further comprise agents which improve the solubility, half-life, absorption, etc. of the antibody. Furthermore, the compositions of the present disclosure may further comprise agents that attenuate undesirable side effects and/or decrease the toxicity of the antibodies(s). Examples of such agents are described in a variety of texts, such a, but not limited to, Remington: The Science and Practice of Pharmacy (20th Ed., Lippincott, Williams & Wilkins, Daniel Limmer, editor).

The compositions of the present disclosure can be administered in a wide variety of dosage forms for administration. For example, the compositions can be administered in forms, such as, but not limited to, injectable solution, lyophilized powder, or granules.

In the present disclosure, the pharmaceutical compositions may further comprise a pharmaceutically acceptable carrier. Such carriers include, but are not limited to, vehicles, adjuvants, suspending agents, inert fillers, diluents, excipients, wetting agents, binders, buffering agents, disintegrating agents and carriers. Typically, the pharmaceutically acceptable carrier is chemically inert to the active antibodies and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration of the ASFV-specific immunoglobulins in solid form, such as but not limited to powders, or granules, the antibodies may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like.

Formulations suitable for parenteral administration include aqueous isotonic sterile injection solutions, which can contain anti-oxidants, buffers, bacteriostats, and solutes that render the formulation isotonic with the blood of the subject, and aqueous suspensions that can include suspending agents, solubilizers, thickening agents, stabilizers, and preservatives. The composition may be administered in a physiologically acceptable diluent, such as a sterile liquid or mixture of liquids, including water, saline, aqueous dextrose and related sugar solutions.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

The compositions of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyleneoxidepolylysine substituted with palmitoyl residues. Furthermore, the antibodies of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

The pharmaceutical compositions of the present disclosure may be modified to prevent adverse reactions in the subject. Such potential adverse reactions include host recognition, anaphylaxis, localized inflammation and other forms of allergic reaction.

Adverse reactions to immunoglobulin compositions are more common in heterologous antibody treatment than in homologous antibody treatment, although the advantages of IgY antibodies in this respect have been explained. In some embodiments of the pharmaceutical composition, the antibody is modified to alter the Fc region of the molecule. In further embodiments, the antibody is treated to prevent binding between the Fc region of the antibody and the Fc receptor of a cell.

The pharmaceutical preparations of the present disclosure can be stored in any pharmaceutically acceptable form, including an aqueous solution, a frozen aqueous solution, a lyophilized powder, or any of the other forms described herein.

Non-limiting examples of the pharmaceutically acceptable ASFV-specific immunoglobulin composition and/or the ASFV-vaccine composition the ASFV-vaccine composition preferably further comprises an anti-inflammatory.

Non-limiting examples of the pharmaceutically acceptable ASFV-specific immunoglobulin composition preferably further comprise an antigen-binding fragment of an antibody such as an Fab or Fab2 fragment, that may substitute for the antibody. For example, the antigen-binding fragment may be any fragment that includes the antigen-binding region of the original IgY. In some embodiments of the compositions and methods, a modified version of an IgY antibody may substitute for the IgY antibody, so long as the antigen-binding region of the IgY antibody retains its ability to recognize ASFV.

Non-limiting examples of the pharmaceutically acceptable ASFV vaccine composition preferably further comprise a composition of lyophilized powder such as for long-term storage and/or transportation. The lyophilized vaccine can be reconstituted into a solution, such as saline, to about the original volume before being used for immunization or vaccination.

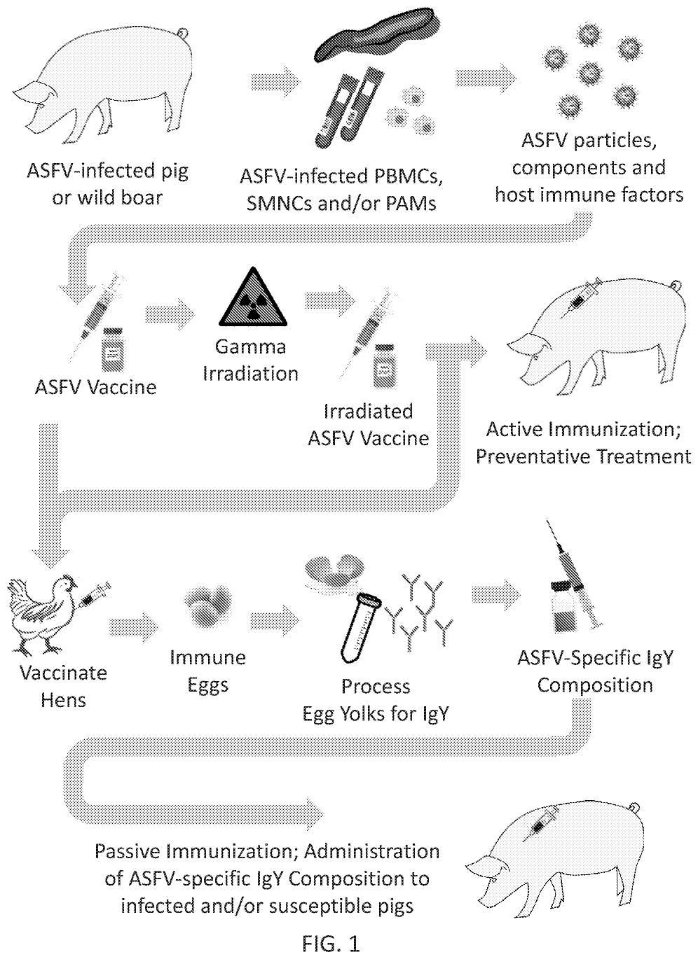

An aspect of the present disclosure is a preferred method for treating ASFV-infected or exposed pigs or wild boars, the method comprised of generating passive immunity in a ASFV-infected or exposed pig or wild boar (FIG. 1). The ASFV-specific immunoglobulin composition may comprise additional components as pharmaceutical components discussed elsewhere in the disclosure. The ASFV-specific immunoglobulin composition may be administered via intraperitoneal or intramuscular injection at a dose of about 0.5 to about 1.0 mg per kg body weight twice a week for one or more weeks an ASFV-infected or exposed pig or wild boar in need thereof.

An aspect of the present disclosure is a method for treating ASFV-infected or exposed pigs or wild boars by administering a composition comprising ASFV-specific immunoglobulins. The ASFV-specific immunoglobulins can be administered orally, at a dose of about 1.0 mg per kg body weight, added to the feed about once per day for about 5 to about 7 consecutive days, to an ASFV-infected or exposed pig or wild boar in need thereof.

Such oral administration methods for ASFV-specific immunoglobulins additionally include the oral administration of the uncooked yolk or yolk-fraction of the egg, alone or in combination with the white of the egg. Oral administration of the raw yolk or yolk-fraction may be performed for example by eating the yolk-fraction. The yolk-fraction or water-soluble yolk fraction may be administered in combination with other ingredients to make it more palatable or nutritious. Thus the yolk-fraction may be consumed by the subject as a food item; alternatively, the yolk-fraction may be consumed as part of a pharmaceutical composition. It is preferably uncooked or very lightly cooked yolk-fraction as cooking can inactivate the antibody.

In one embodiment the water-soluble fraction of the egg yolk can be readily mixed with food of any type or any edible ingredient. The compositions can also be formulated to contain or provide a portion of the macronutrient and micronutrient requirements for an animal, and can be provided as a replacement for, or a supplement to, the animal's regular diet. The composition can be provided as, added to, or mixed with a snack, treat, chew, or other supplement to the normal intake of food.

Non-limiting examples of the method of treatment include an increased dose of ASFV-specific immunoglobulins administered either parenterally or orally in combination with or alternatively, administered at an increased dosing frequency. An aspect of the present disclosure is a preferred method for treating pregnant sows, the sow's fetuses, and/or piglets of ASFV-infected or exposed pigs or wild boars. ASFV-specific immunoglobulins are administered to the pregnant sows by methods discussed elsewhere in the disclosure. The piglets and/or pig fetuses directly or indirectly receive the ASFV vaccine during gestation and/or nursing.

In another aspect of the present disclosure is a preferred preventative method of treatment for pigs or wild boars susceptible to ASF infection (FIG. 1). The irradiated ASFV vaccine compositions can be preferably administered to subjects (i.e., pigs or wild boars), including but not limited to the following, subjects which have been exposed to ASFV, subjects that are susceptible to ASF infection, and/or subjects that are infected with ASFV. The ASFV vaccine composition may comprise additional components such as pharmaceutical components discussed elsewhere in the disclosure. The ASFV vaccine composition may be administered to a subject via intraperitoneal, subcutaneous, or intramuscular injection at a dose of about 0.05 mg/dose to about 1.0 mg/dose, for a younger (i.e., not old) pig of approximately 20 kg body weight. Preferably, the ASFV vaccine composition is administered at a dose of approximately 100 μg. Additionally or alternatively, the ASFV vaccine composition may be administered more than once time to an individual subject. For example, the immunization can be boosted one time 14 days following the first or primary immunization. In addition, a third immunization may be performed at 21 days after the first or primary immunization.

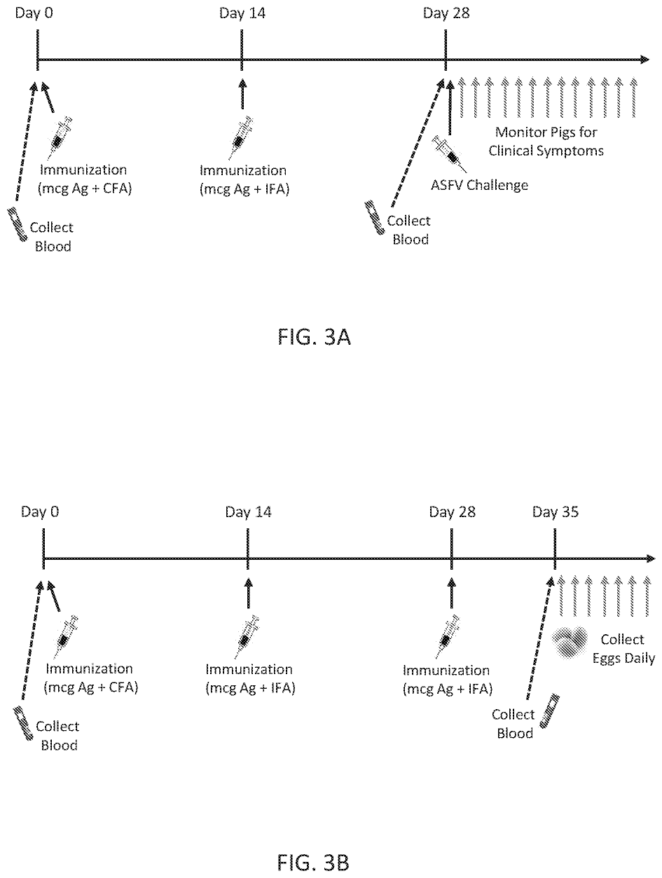

Disclosed herein is an example embodiment diagrammed in FIG. 3A, specifically a method of treating a subject by administering the first dose of the ASFV vaccine composition in a 1:1 ratio with CFA. Then, after about two weeks, a second dose of the ASFV vaccine composition in a 1:1 ratio with IFA can be administered to the subject. About four weeks after the first or primary immunization, the pig or wild boar may be subjected to a ASFV challenge, to determine if the immunized subject can survive a lethal ASF infection. The irradiated ASFV vaccine can be dosed at a range equivalent to about 10⁴ HAD50 (50% hemadsorption dose) to about 105 HAD50 of live viruses.

### EXAMPLES

The following non-limiting examples support the concept of using the pharmaceutically acceptable ASF vaccine composition for generation of antibodies to be used for treatment of infected pigs and/or wild boars or for prevention of infection of pigs and/or wild boars.

### Example 1

Live ASFV Vaccine Compositions from ASFV Infected Immune Cells

Fresh spleens from ASFV infected pigs were collected and 10 g of the spleens were transferred to Petri dishes with metal mesh. Using the metal mesh, the spleen tissue was minced and single cells were collected. Contaminated RBCs were lysed using 0.83% NH₄Cl. SMNCs were washed with cold PBS and subjected to two freeze-thaw cycles. The SMNC lysate was centrifuged and the supernatant was collected.

Peripheral blood (40 ml) was collected from ASFV infected pigs into Ethylenediaminetetraacetic acid (EDTA)-treated blood collection tubes and subjected to centrifugation. The buffy coat, containing white blood cells or PBMCs, was collected. The PBMCs were washed with cold PBS and subjected to two freeze-thaw cycles. The PBMC lysate was centrifuged and the supernatant was collected.

Next, alveolar macrophages from healthy, uninfected pigs were collected and cultured in medium without serum (FIG. 2A). The following day, the PAMs were infected with ASFV stock. The ASFV infected PAMs were cultured for 7 days. A cytopathic effect on the PAMs was observed at 3 days post-ASFV infection (FIG. 2B), at 4 days post-ASFV infection (FIG. 2C), and at 7 days post-ASFV infection (FIG. 2D). The entire content of ASFV infected PAM culture (approximately 2× 10⁸ cells), including cells and culture medium, was collected and subjected to two freeze-thaw cycles, centrifuged, and supernatant was collected.

Virus titers of the supernatants from the SMNCs, PBMCs, and PAMs lysates were determined using a hemadsorption test. The lysates were mixed equally based on 50% hemadsorption dose (HAD50), yielding live ASFV vaccine compositions. The live ASFV vaccine compositions, derived from SMNCs, PBMCs, and PAMs were analyzed for protein concentration (Table 1).

TABLE 1
Live ASFV Vaccine Compositions

| Vaccine derived from: | SMNCs | PBMCs | PAMs |
|---|---|---|---|
| Volume (ml) | 50 | 100 | 20 |
| Protein Concentration (mg/ml) | 0.33 | 0.3 | 0.67 |
| Virus Titers (log₁₀HAD₅₀/ml) | 6.18 | 6.49 | 6.14 |

### Example 2

### Inactivated ASFV Vaccine Compositions

First, fresh spleen and lungs from pigs with severe ASFV infection were collected and homogenized in cold PBS using a tissue homogenizer. The homogenates were subjected to two freeze-thaw cycles and centrifuged. The supernatant from ASFV-infected tissue was collected, the virus titers were determined, and the supernatant was used to prepare a live ASFV vaccine composition, as described in Example 1.

The live ASFV vaccine composition derived from ASFV-infected tissue (fresh spleen and lungs from pigs with severe ASFV infection), as well as the live ASFV vaccine compositions derived from the SMNC, PBMC, and PAM lysates, described in Example 1, were inactivated by subjecting the compositions to gamma-irradiated using 6Co irradiator.

The gamma-irradiated ASFV vaccine compositions were added to healthy PAM cultures. Complete ASFV inactivation of the gamma-irradiated ASFV vaccine compositions was confirmed when the PAMs did not show hemadsorption or cytopathic effects after more than 7 days in culture. The gamma-irradiated ASFV vaccine compositions were also injected into healthy pigs, which did not develop ASF symptoms.

### Example 3

Immunization of Hens with Live ASFV Vaccine Compositions

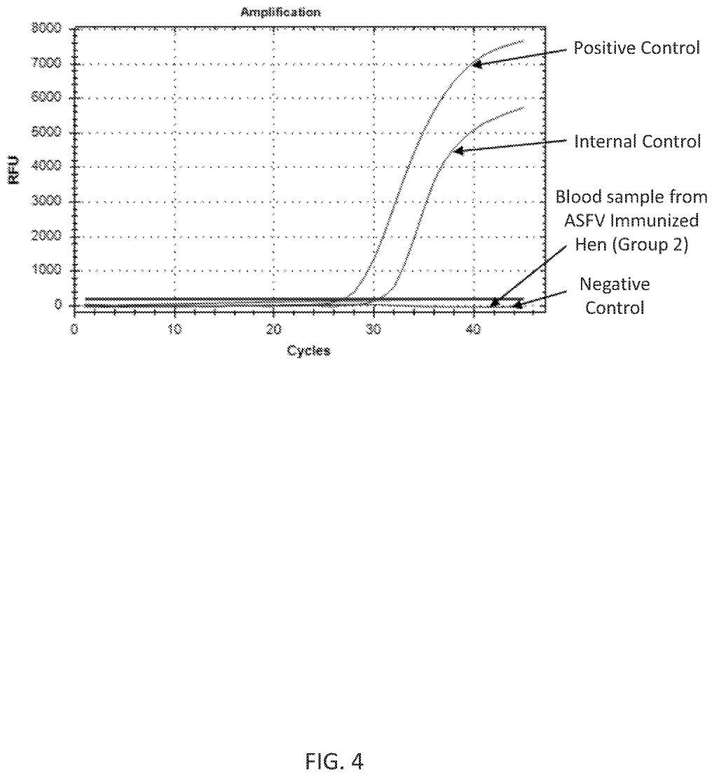

Three groups of hens (n=3 per group) were immunized with live ASFV vaccine on day 1, day 14, and on day 28. Group 1 received saline as control (no vaccine), Group 2 received ASFV vaccine Formulation 1, containing whole ASF virus particles, ASF viral components, and immunosuppressive protein factors derived from infected spleen, and Group 3 received ASFV vaccine Formulation 2 comprising of whole ASF virus particles, ASF viral components, and immunosuppressive protein factors derived from infected spleen and peripheral blood. Following the second and third immunization, blood samples were collected and analyzed for the presence of ASFV DNA using qPCR. No ASFV DNA was detected in the blood samples from chickens previously immunized with the live ASFV vaccine composition, confirming there was no viral shedding in the immunized hens. FIG. 4 is a representative qPCR graph showing no ASFV DNA in a blood sample from an immunized hen in Group 2.

### Example 4

### Gamma Irradiation of ASFV Proteins

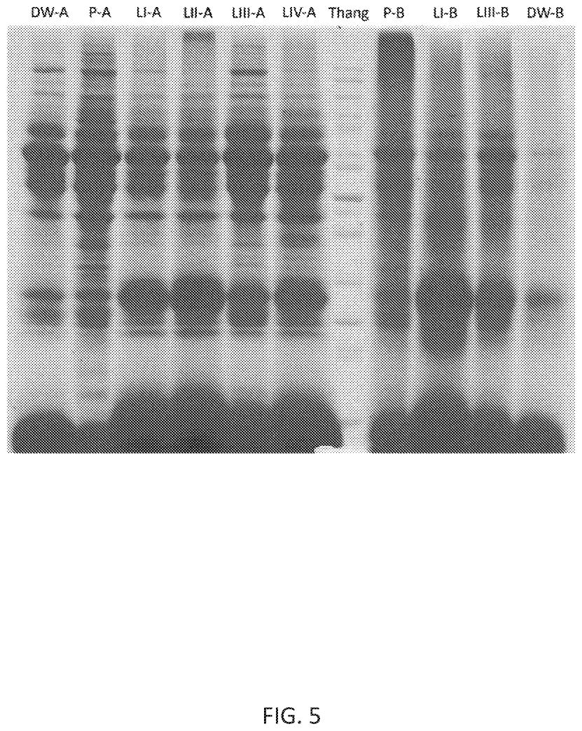

ASFV proteins derived from ASFV-infected tissue were isolated and subjected to gamma irradiation. FIG. 5 shows the damaging effects of a high dose of gamma irradiation (i.e., 25 kGy) on ASFV proteins. Gel electrophoresis reveals the effects of 25 kGy irradiation on ASFV proteins (50 mg/lane; lanes 8-11) vs. unirradiated ASFV protein samples (50 mg/lane; lanes 1-6); ladder is shown in lane 7 (Thang), top molecular marker band is 200 kDa and the lower band is 10 kDa. The dose of gamma irradiation is critical to the viability of the live ASFV vaccine (see FIG. 5). Experiment 4 shows that gamma irradiation doses should be less than 25 kGy, preferably no more than about 20 kGy, as higher doses of gamma irradiation are not viable for the ASFV vaccine to generate antibodies because they alter the structure of ASFV proteins.

### Example 5

### Generation of ASFV-Specific Immunoglobulins

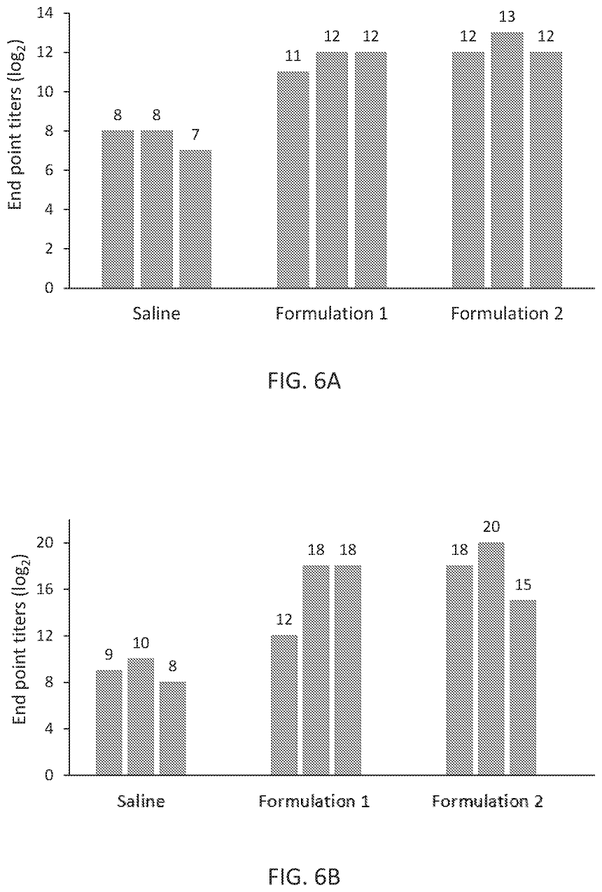

Eggs were collected from the immunized hens described in Example 3, after the second and third immunizations. IgY Immunoglobulins were extracted from egg yolks using a simple water dilution method. These immunoglobulin compositions were then analyzed for ASFV-specific antibody titers using recombinant ASFV major capsid protein p72-coated ELISA plates (SEQ ID NO: 2). The results of Example 5, as shown in FIG. 6, demonstrate that chickens immunized with live ASFV vaccine compositions generate IgY antibody pools with comprehensive specificities to ASFV components, such as the ASFV major capsid protein p72 (SEQ ID NO: 2), after 14 days (FIG. 6A) and after 28 days (FIG. 6B).

### Example 6

A Single Dose of ASFV-Specific Immunoglobulins Delayed ASF Symptom Onset and Prolonged Survival of Pigs with Severe ASFV Infection

An ASFV vaccine composition was prepared from a homogenate of ASFV-infected spleen and ASFV-infected buffy coat containing PBMCs from an ASFV-infected pig. The PBMC mixture was frozen in a dry ice ethanol bath and thawed to room temperature. The freeze-thaw procedures was repeated two times. Three groups of egg-laying hens (n=3/group) were administered control or 1 of 2 different formulations of ASFV vaccine. Group 1 received saline as control (no vaccine), Group 2 received ASFV vaccine Formulation 1 (prepared from spleen homogenate), and Group 3 received ASFV vaccine Formulation 2 (prepared from spleen homogenate). The hens were actively immunized by administering the ASFV vaccine compositions (via intramuscular injection), or given control, on day 1, day 14, and day 30. Blood samples were taken from the chickens after the second immunization on day 14 and qPCR confirmed there was no virus shedding.

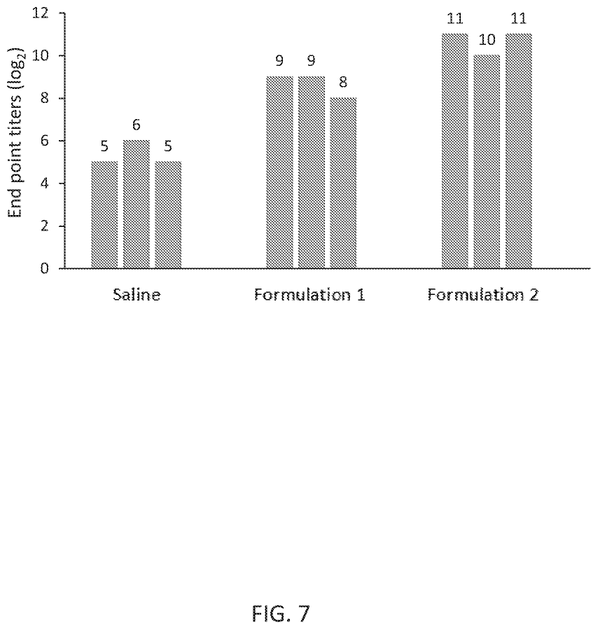

Eggs were collected daily following the third immunization. IgY Immunoglobulins were extracted from egg yolks using a simple water dilution method. ASFV-specific antibody titers were analyzed as previously described in Example 5; results are shown in FIG. 7.

Due to the high ASFV-specific antibody titers, eggs collected from the hens that received Formulation 2 of the ASFV vaccine composition were used to prepare the ASFV-specific immunoglobulin composition to be administered to pigs.

Three groups of adult pigs were designated as A, B, and C. Group A was made up of 6 adult pigs (approximate 20 kg each), and received 100 mg of ASFV-specific immunoglobulin composition one day before being exposed to ASFV (day 1). Group B was made up of 3 adult pigs, which received 100 mg of ASFV-specific immunoglobulin composition one day after exposure to ASFV (day 3). Lastly, Group C was made up of 3 adult pigs, exposed to ASFV and did not receive the ASFV-specific immunoglobulin composition. All three groups of pigs were subjected to a high dose of ASFV, approximately 105 live contagious virus particles (day 2), which generated a severe ASFV infection.

Clinical observations revealed that treatment with ASFV-specific immunoglobulins delayed symptom onset after ASFV exposure. The untreated pigs (Group C) began showing initial ASF symptoms on day 6, including reduced activity (i.e., lethargy), a reduction in appetite (i.e., decreased food consumption), and shortness of breath (i.e., laboured breathing). Symptoms quickly worsened and by day 8, all three pigs from Group C had stopped eating. Pigs that received ASFV-specific immunoglobulins one day after severe ASFV infection (Group B), experienced a delay in symptom onset, displaying initial ASF symptoms on day 8. An even greater delay in ASF symptom onset was observed in pigs that received ASFV-specific immunoglobulins one day before severe ASFV infection (Group A). Group A pigs revealed initial ASF symptoms on day 10.

In addition to delaying symptom onset following severe ASFV infection, treatment with ASFV-specific immunoglobulins also prolonged survival. The mean survival day for Group C pigs was day 9, with the last pig of Group C expiring on day 11. Pigs treated with ASFV-specific immunoglobulins experienced prolonged survival compared to untreated pigs, with a mean survival of day 13. The last pig from Group A and Group B survived until day 17.

Results revealed that administration of the ASFV-specific immunoglobulin composition either before or after severe ASFV infection generated passive immunity, delayed ASF symptom onset, and prolonged survival.

1. A method of treating African swine fever (ASF) viral (ASFV) infection in an infected pig or wild boar, the method comprising:
administering a first composition to an egg-laying fowl, the first composition comprising one or more immunosuppressive protein factors and further comprising ASF virus particles and/or ASF viral components; and
providing a second composition to the infected pig or wild boar, the second composition comprising a water-soluble fraction of egg yolk from the egg-laying fowl who has been administered the first composition.

2. The method of claim 1, wherein the second composition is administered to the infected pig or wild boar in an amount that provides a dose of the water-soluble fraction of egg yolk that is about 0.05 mg to about 1.0 mg per kg body weight of the infected pig or wild boar.

3. The method of claim 1, wherein the second composition is administered to the infected pig or wild boar for a time period comprising at least once per week or 7 consecutive days.

4. The method of claim 1, wherein the second composition is administered to the infected pig or wild boar parenterally by intramuscular or intraperitoneal injection.

5. The method of claim 1, wherein the second composition is a food product administered orally.

6. (canceled)

7. (canceled)

8. (canceled)

9. (canceled)

10. The method of claim 1, where the ASF virus particles and ASF viral components are inactivated by gamma irradiation at a gamma irradiation dose of about 15 kGy to about 20 kGy.

11. The method of claim 1, wherein the ASF virus particles, ASF viral components, and/or immunosuppressive protein factors are derived from ASFV-infected spleen mononuclear cells (SMNCs), ASF-infected peripheral blood and mononuclear cells (PBMCs), and/or ASF-infected primary alveolar macrophages (PAMs).

12. A method of preventing, decreasing incidence of, and/or decreasing severity of ASF viral infection in a pig or wild boar at risk thereof, the method comprising:
administering a first composition to an egg-laying fowl, the first composition comprising one or more immunosuppressive protein factors and further comprising ASF virus particles and/or ASF viral components; and
providing a second composition to the pig or wild boar, the second composition comprising a water-soluble fraction of egg yolk from the egg-laying fowl who has been administered the first composition.

13. The method of claim 12, wherein the second composition is administered to the pig or wild boar in an amount that provides a dose of the water-soluble fraction of egg yolk that is about 0.05 mg to about 1.0 mg per kg body weight of the infected pig or wild boar at risk thereof.

14. The method of claim 12, wherein the second composition is administered to the pig or wild boar for a time period comprising at least once per week or 7 consecutive days.

15. (canceled)

16. (canceled)

17. (canceled)

18. (canceled)

19. (canceled)

20. (canceled)

21. (canceled)

22. The method of claim 12, where the ASF virus particles and ASF viral components are inactivated by gamma irradiation at a gamma irradiation dose of about 15 kGy to about 20 kGy.

23. A method of producing an orally administrable composition effective to treat, prevent, decrease incidence of, and/or decrease severity of African swine fever (ASF) viral (ASFV) infection in an infected pig or wild boar, the method comprising adding a water-soluble fraction of egg yolk, which has been obtained from an egg-laying fowl who has been administered one or more immunosuppressive protein factors and ASF virus particles and/or ASF viral components, to at least one other edible ingredient to thereby form the orally administrable composition.

24. The method of claim 23, wherein the ASF virus particles, ASF viral components, and/or immunosuppressive protein factors are derived from ASF-infected spleen mononuclear cells (SMNCs), ASF-infected peripheral blood and mononuclear cells (PBMCs), and/or ASF-infected primary alveolar macrophages (PAMs).

25. (canceled)

26. The method of claim 23, where the ASF virus particles and ASF viral components are inactivated by gamma irradiation at a gamma irradiation dose of about 15 kGy to about 20 kGy.

27. (canceled)

28. (canceled)

29. (canceled)

30. A unit dosage form comprising a therapeutically or prophylactically effective amount of a composition comprising a water-soluble fraction of egg yolk, which has been obtained from an egg-laying fowl who has been administered one or more immunosuppressive protein factors and ASF virus particles and/or ASF viral components.

31. The unit dosage form of claim 30, wherein the ASF virus particles, ASF viral components, and/or immunosuppressive protein factors are derived from ASF-infected spleen mononuclear cells (SMNCs), ASF-infected peripheral blood and mononuclear cells (PBMCs), and/or ASF-infected primary alveolar macrophages (PAMs).

32. (canceled)

33. The unit dosage form of claim 30, wherein the where the ASF virus particles, ASF viral components, and/or immunosuppressive protein factors are inactivated by gamma irradiation at a gamma irradiation dose of about 15 kGy to about 20 kGy.

34. (canceled)

35. (canceled)

36. (canceled)

37. The method of claim 1, wherein one or more immunosuppressive protein factors comprise cytokine comprising (i) TNF cytokines and/or (ii) pro-inflammatory cytokines comprising IL-17F, and/or interferons.

38. The method of claim 1, wherein one or more immunosuppressive protein factors comprise interleukins, chemokines, colony-stimulating factors, and/or cytokines involved in the cytokine storm.

39. The method of claim 1, wherein one or more immunosuppressive protein factors comprise TNF-α, IFN-α, IL-1β, IL-6, IL-8, IL-12, IL-18, and/or RANTES.

40. The method of claim 12, wherein one or more immunosuppressive protein factors comprise cytokines comprising (i) TNF cytokines and/or (ii) pro-inflammatory cytokines comprising IL-17F, and/or interferons.

41. The method of claim 12, wherein one or more immunosuppressive protein factors comprise interleukins, chemokines, colony-stimulating factors, and/or cytokines involved in the cytokine storm.

42. The method of claim 12, wherein one or more immunosuppressive protein factors comprise TNF-α, IFN-α, IL-1β, IL-6, IL-8, IL-12, IL-18, and/or RANTES.

43. The method of claim 23, wherein one or more immunosuppressive protein factors comprise cytokines comprising (i) TNF cytokines and/or (ii) pro-inflammatory cytokines comprising IL-17F, and/or interferons.

44. The method of claim 23, wherein one or more immunosuppressive protein factors comprise interleukins, chemokines, colony-stimulating factors, and/or cytokines involved in the cytokine storm.

45. The method of claim 23, wherein one or more immunosuppressive protein factors comprise TNF-α, IFN-α, IL-1β, IL-6, IL-8, IL-12, IL-18, and/or RANTES.

46. The unit dosage form of claim 30, wherein one or more immunosuppressive protein factors comprise cytokines comprising (i) TNF cytokines and/or (ii) pro-inflammatory cytokines comprising IL-17F, and/or interferons.

47. The unit dosage form of claim 30, wherein one or more immunosuppressive protein factors comprise interleukins, chemokines, colony-stimulating factors, and/or cytokines involved in the cytokine storm.

48. The unit dosage form of claim 30, wherein one or more immunosuppressive protein factors comprise TNF-α, IFN-α, IL-1β, IL-6, IL-8, IL-12, IL-18, and/or RANTES.

49. (canceled)

50. (canceled)
